(19) United States
(12) Patent Application Publication
(10) Pub. No.: US 2013/0289562 A1
(43) Pub. Date: Oct. 31, 2013

(54) Bi-Polar Ablation Devices and Methods of Use

(71) Applicants: James Skarda, Lake Elmo, MN (US); William Wenger, St. Paul, MN (US)

(72) Inventors: James Skarda, Lake Elmo, MN (US); William Wenger, St. Paul, MN (US)

(73) Assignee: Medtronic, Inc., Minneapolis, MN (US)

(21) Appl. No.: 13/458,146

(22) Filed: Apr. 27, 2012

(51) Int. Cl. A61B 18/12

(52) U.S. Cl. 606/51

(57) ABSTRACT

An ablation device for use in a surgical procedure, including a first member having a first elongated shaft and a first jaw extending from a distal end of the first elongated shaft, and a second member having a second elongated shaft and a second jaw extending from a distal end of the second elongated shaft, wherein the second elongated shaft is at least partially positioned within an inner tubular opening of the first elongated shaft, and wherein the second jaw has a bottom surface that is generally parallel to at least a portion of a top surface of the first jaw. The ablation device further includes a handle extending from a proximal end of the first elongated shaft and a finger grip extending radially outwardly relative to an outer surface of the first elongated shaft.

### TECHNICAL FIELD

The present invention relates generally to surgical tools and procedures, and more particularly to the use of electrosurgical ablation for treatment of tissue.

### BACKGROUND

In patients with chronic atrial fibrillation or having atrial tachycardia that is resistant to medical treatment, certain treatment procedures have been used that control propagation of depolarization wavefronts in the right and left atria. Such procedures can include surgical incisions through the walls of the right and left atria, which create blind or dead end conduction pathways that prevent re-entrant atrial tachycardias from occurring. While such procedures can be successful in treating atrial fibrillation, the procedure can be quite complex and therefore be practiced by only a few very skilled cardiac physicians in conjunction with other open-heart procedures. The procedure also is relatively traumatic to the heart, as the right and left atria are essentially severed and then sewed back together in order to define lines of lesion across which the depolarization wavefronts will not propagate.

As an alternative to the incisions described above, electrosurgical ablation procedures can be used, for example, by applying radiofrequency (RF) energy to internal or external surfaces of the atria to create lesions across which depolarization wavefronts will not propagate. In order for such procedures to be effective, it is desirable that the electrosurgically created lesions are continuous along their length and extend completely through the tissue of the heart (i.e. transmural lesions). These goals may be difficult to accomplish employing dry ablation electrodes or electrodes applied only to the interior or exterior surfaces of the heart tissue. Therefore, certain electrosurgical hemostats are configured to allow fluid-assisted tissue ablation, such as those generally described in U.S. Pat. No. 6,096,037 (Mulier), which is incorporated herein by reference in its entirety.

Certain fluid-assisted tissue ablation systems include jaws at a distal end for providing ablative treatment of tissue, where control mechanisms for these jaws are typically located at a proximal end of the device, such as at a handle. In some cases, the distance between the proximal and distal ends of the system can be relatively large and the system can therefore include at least one malleable or articulating component to allow the surgeon to customize the system for each particular patient. While such systems can be effective for many situations, there is a desire to provide additional ablation systems that provide for a smaller distance between distal jaws and the control end of the system to provide surgeons with additional alternative devices for accessing areas of the patient's body.

### SUMMARY

In one aspect of the invention, bipolar parallel jaw sub-assemblies are provided that can be used in different orientations on a bipolar ablation device. Such jaw assemblies can be used as an independent clamp in a concomitant procedure. Alternatively, the jaw assemblies can be extended with a rigid or malleable neck to expand their versatility to different types of procedures. The jaws of such devices can operate in a mechanical channel for a manner of sliding in a distal and proximal direction. In another aspect of the invention, a protected sheath drive cable can be used to provide a level of flexibility and also to provide enough force to allow the jaws to clamp the tissue sufficiently. Such devices may optionally include an internal spring to keep the jaws in a closed position. In yet another aspect of the invention, a stand-alone jaw set could be clipped onto tissue in a similar manner to that of a clothespin during open chest concomitant procedures. In another aspect of the invention, bipolar ablation jaws are provided with parallel closure as a subassembly, which provides many options for bipolar ablation procedural adaptability. With any or all of these embodiments, it is possible for the system to include a quick connect feature which allows the user to easily exchange components, such as to provide devices with differing neck lengths, styles, flexibility, rigidity, angles, or other features. It is further contemplated that the jaw subassemblies can be used as a monopolar device.

In another aspect of the invention, a relatively rigid bipolar direct drive device is provided, which includes a malleable section with an internal incompressible coil. Such a bipolar device can be used in a concomitant ablation procedure to treat atrial fibrillation. A device can include the capability to mechanically drive the jaws and still remain malleable on the outside. Such a device can be relatively small in size and can be configured to allow for sufficient visualization of the treatment area. This can be accomplished, for example, by using a handle design that has a low profile and includes a malleable section that allows more degrees of freedom for device placement and for spacing the surgeon's hand from the ablation site. In one embodiment, the device includes a direct drive assembly that has a one to one mechanical advantage as the jaws are actuated. The device includes an internal incompressible coil that is slightly longer than a malleable neck section in order to allow the used to shape the neck with a particular radius, while still having direct drive actuation of the jaws. The internal incompressible coil may have a liner (e.g., a PTFE liner) over the coil.

### BRIEF DESCRIPTION OF THE DRAWINGS

The present invention will be further explained with reference to the appended Figures, wherein like structure is referred to by like numerals throughout the several views, and wherein:

FIG. 1 is a front view of an embodiment of an ablation device of the invention;

FIG. 2 is an exploded view of the ablation device illustrated in FIG. 1;

FIG. 3 is a front view of an embodiment of an ablation device of the invention;

FIG. 4 is a front view of an embodiment of an ablation device, including attached fluid tubing and electrical wiring;

FIG. 5 is a front view of an embodiment of an ablation device in a relaxed configuration with the jaws in a naturally closed position;

FIG. 6 is a front view of the ablation device of FIG. 5 in an activated configuration with the jaws moved to an open position;

FIG. 7 is a perspective view of a proximal end portion of an ablation device in a relaxed configuration;

FIG. 8 is a perspective view of the proximal end portion of an ablation device of FIG. 7 as it is being activated with a hand;

FIG. 9 is a schematic perspective view of a finger flange that is adaptable for use with ablation devices of the invention;

FIG. 10 is a front view of another embodiment of an ablation device being activated by a hand;

FIGS. 11a-11c are front, perspective, and front views, respectively, of an embodiment of an ablation device that optionally includes finger grips and finger wraps to secure the device to fingers of the operator;

FIG. 12 is a front view of an ablation device of the invention;

FIG. 13 is a front view of another ablation device of the invention;

FIG. 14 is an exploded view of the ablation device illustrated in FIG. 13;

FIG. 15 is an enlarged view of a handle of an ablation device of the invention;

FIG. 16 is a front view of an ablation device of the invention with an electrical cord extending from its proximal end;

FIG. 17 is a front view of another embodiment of an ablation device of the invention;

FIG. 18 is a front view of another embodiment of an ablation device of the invention;

FIG. 19 is an exploded view of the ablation device illustrated in FIG. 18;

FIG. 20 is a front view of another embodiment of an ablation device of the invention;

FIG. 21 is a front view of another embodiment of an ablation device of the invention;

FIG. 22 is a front view of another embodiment of an ablation device of the invention;

FIG. 23 is a front view of another embodiment of an ablation device of the invention, illustrating a quick connect/disconnect feature;

FIG. 24 includes a front view and an exploded view of another embodiment of an ablation device of the invention;

FIG. 25 is a perspective view of a clip-on sensing apparatus of the invention;

FIG. 26 is a front view of a clip-on sensing apparatus positioned relative to an ablation device; and

FIG. 27 is a perspective view of a portion of an ablation device with a clip-on sensing apparatus positioned on one of the distal jaws of the ablation device.

### DETAILED DESCRIPTION

The present invention relates generally to devices and systems that can be used in open-chest (i.e., sternotomy) concomitant bipolar ablation surgical procedures. These ablation devices can include a dual linear electrode device that provides integral saline delivery to both electrodes. These devices are generally capable of rapidly creating linear transmural lesions in both atria of the heart during open chest cardiac procedures to reproduce certain lesion patterns on the heart.

In certain embodiments, the devices are relatively small and are capable of reaching the desired lesion locations with minimal steering or complicated maneuvering. Some of the devices disclosed herein are therefore somewhat smaller and shorter than known devices that are used for bipolar surgical procedures. These relatively small devices can be held in the palm of a surgeon's hand, for example, and can therefore serve as a sort of extension of the surgeon's hand for accessing the lesion areas. In many embodiments, the device includes two wires or tubes that are used for activation of the device, which include a tube that connects to a saline bag and an electric wire or connector that can be connected to a radio frequency (RF) generator. The device can optionally include an EPROM within its connectors to prevent re-use of the device.

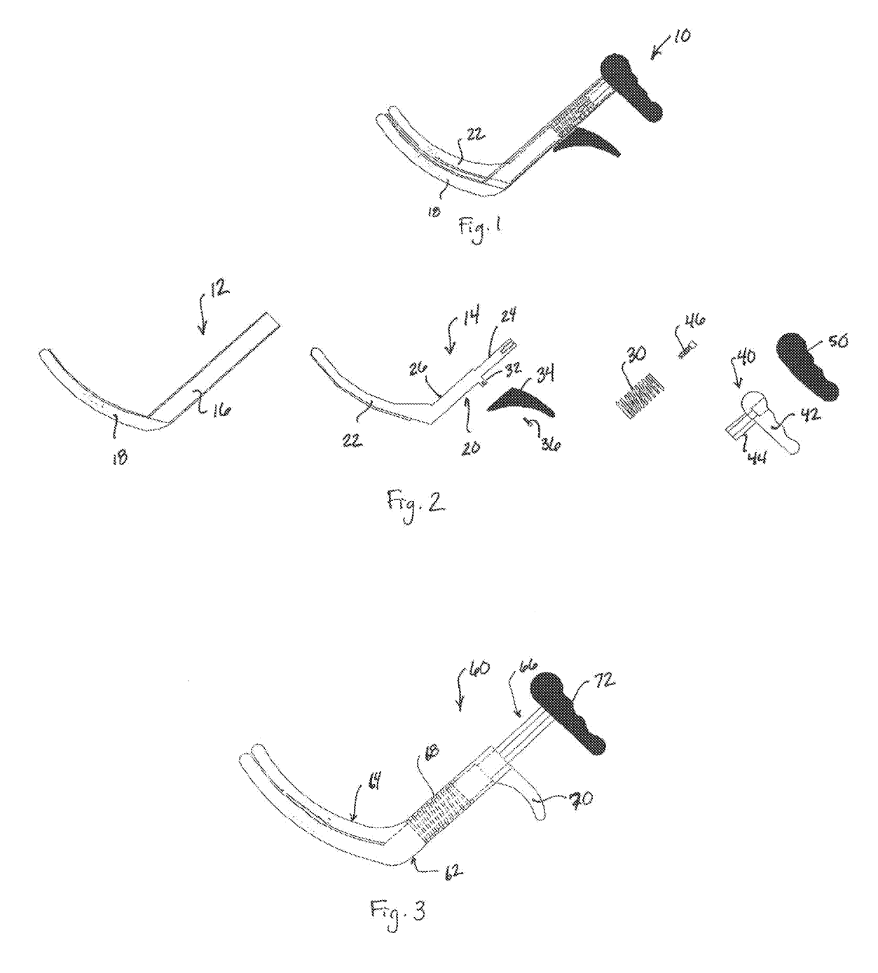

Referring now to the Figures, wherein the components are labeled with like numerals throughout the several Figures, and initially to FIGS. 1 and 2, an exemplary configuration of an ablation device 10 is illustrated, which can be connected to components such as electrical and fluidic connections, as described generally above, although such components are not illustrated in these figures. Ablation device 10 includes a first member 12, and a second member 14 that is linearly moveable relative to the first member 12. In particular, first member 12 includes an extension member 16, which is a generally elongated tubular shaft, from which a first or lower jaw 18 extends, while second member 14 includes an extension member 20, which is generally an elongated shaft, from which a second or upper jaw 22 extends. Extension member 20 of second member 14 includes a portion 24 that has a smaller cross-section than the portion 26 that extends directly from the upper jaw 22. In this way, when the device 10 is assembled, a spring 30, which has an inner diameter that is at least slightly smaller than an outer diameter of portion 26 but slightly larger than an outer diameter of portion 24, can be slid over the portion 24 until it reaches portion 26. The spring 30 will then be unable to slide further along extension member 20 toward the jaw 22, thereby maintaining the spring in this position relative to second member 14. Second member 14 further includes an attachment post 32 to which a finger grip 34 can be attached, as explained in further detail below

Second member 14 is positioned relative to first member 12 so that its extension member 20 is generally co-linear and at least partially enclosed within a center lumen of extension member 16. Extension member 16 includes an opening (not visible), such as a slot, that extends along a portion of its length and is sized to allow the finger grip 34 to be attached to the attachment post 32 that extends through it, such as with a fastener 36. In order to assemble the device, a handle 40, which includes a thumb rest 42 from which a post 44 extends, is attached to the first member 12 via the post 44. The device may optionally include a cover 50 over at least a portion of the thumb rest 42.

To assemble the components of the device, a fastener 46 can be inserted into an opening of the thumb rest 42, such as by screwing the fastener 46 into an opening in the handle portion 40. The second member 14 can move in a proximal and a distal direction along the first member 12, along with the spring 30, which can be a coil spring. Spring 30 controls the force at which the lower jaw 18 and upper jaw 22 can close together and also defines the force that needs to be overcome in order to move the jaws 18, 22 away from each other and into an open position. Therefore, the spring 30 can be chosen and/or designed to provide a desired actuation force for the user to move the jaws relative to each other.

The jaws 18, 22 of ablation device 10 are in a normally closed position when the device 10 is not subjected to external forces, with the jaws 18, 22 being generally parallel to each other along at least a portion of their surfaces that face each other. That is, a bottom face of jaw 22 is preferably in contact with at least a portion of an top face of jaw 18 when the jaws are closed. In order to move these jaws away from each other, a user can place a thumb on the thumb rest 42 and a finger on a curved surface of finger grip 34, and then squeeze the finger grip 34 toward the thumb rest 42 to slide the second member 14 toward the thumb rest 42, thereby moving the jaws 18, 22 away from each other. When this movement is occurring, the spring 30 will be compressed by a corresponding amount. The spring 30 may be made of spring steel, for example, and may optionally be coated, such as with a PTFE coating.

The ablation device 10 can be a part of a larger system that includes an RF electrode, which may include a porous polymer, hypo-tube, wires, one or more saline lines, and/or strain-relief members. The various components of the device 10 may include machined components, injection molded components, or components that are made using a variety of different manufacturing techniques. The device 10 may further include additional pins, fasteners, and/or hinges in order to maintain the components in alignment with each other during the ablation process.

FIG. 3 illustrates another embodiment of an ablation device 60 that is similarly configured to the embodiment of FIGS. 1 and 2. Ablation device 60 includes a first member 62 and a second member 64, each of which includes a jaw extending at a distal end from an elongated member. The elongated member of second member 64 is positioned generally within the elongated member of first member 62, and a finger grip 70 extends from the first member 62. The device 60 further includes a handle 66 with a thumb rest 72 and a spring 68 positioned between first and second members 62, 64. In order to move the jaws relative to each other, a finger is placed under the curved surface of the finger grip 70, a thumb or palm is placed on the thumb rest 72, and then the finger and thumb are squeezed toward each other to pull the jaws apart, while the spring 68 provides resistance to the movement.

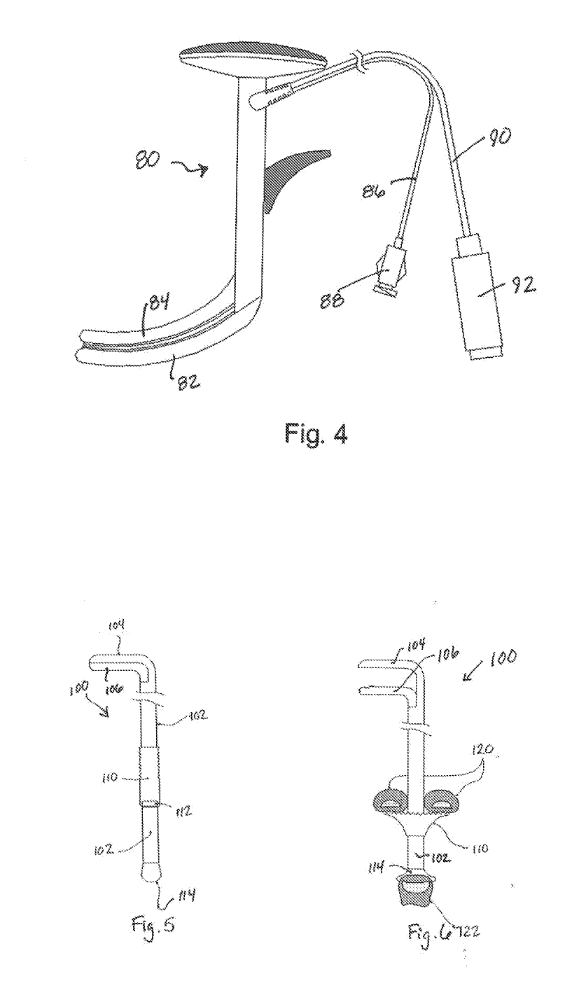

FIG. 4 illustrates an embodiment of an ablation device 80 that is generally configured with the same basic components as the ablation device 10 of FIGS. 1 and 2, including jaw portions 82, 84. This embodiment also is provided in a normally closed position such that a spring force needs to be overcome in order to move the jaws away from each other. These configurations of the jaws in both embodiments are intended to be illustrative, and it is understood that the jaws can be provided with a wide variety of configurations, and may be rigid or malleable. With any configuration of the jaws, however, the surfaces of the jaws that contact each other when the device is in a closed position will generally match or meet each other across some or all of their contact surfaces.

Ablation device 80 includes a first line or tube 86 that fluidly connects the device 80 to a saline source 88, such as a saline bag, and a second line 90 that is an electrical wire connector, which can be connected to a radio frequency (RF) generator 92, for example. The tube 86 and electrical line 90 may be attached to the device 80 at any location along its length that does not interfere with the surgeon's ability to move the jaws during the ablation process. The tube 86 may be a PVC or silicone saline line, for example, and may optionally include a coating, such as parylene. The electrical line 90 may be a 48 AWG twisted wire pair, for example, which can optionally include a lubricous outer coating. Such an electrical line 90 can be selected to be of a type that is resistant to kinking during movement of the upper jaw relative to the lower jaw.

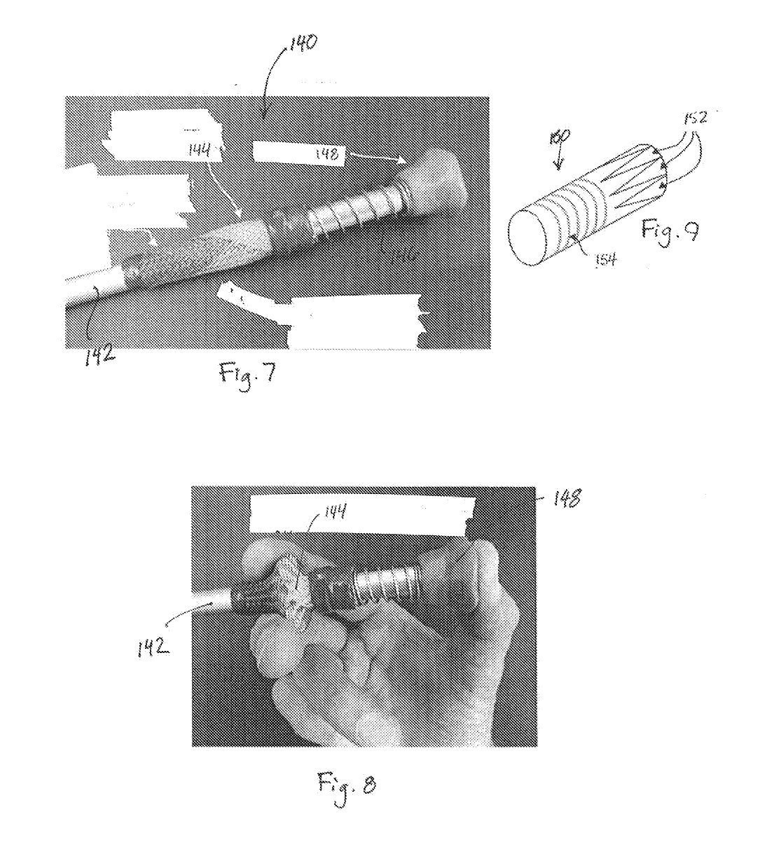

FIGS. 5 and 6 illustrate another embodiment of an ablation device 100 in its closed and open positions, respectively. This device 100 provides a syringe-like style of handle actuation. Ablation device 100 generally includes an outer shaft 102 that extends along the length of the device 100, from which a first distal jaw 104 extends. An inner shaft is not visible in these Figures, but such a shaft also extends along the length of the device 100, and is positioned within the outer shaft 102. A second distal jaw 106 extends from the distal end of the inner shaft and is moveable relative to the first distal jaw 104. Ablation device 100 further includes an elastomeric finger flange 110 that is slideable relative to the outer surface of outer shaft 102. Finger flange 110 is illustrated in its relaxed or uncompressed state in FIG. 5. The ablation device 100 further includes a traveling collar 112 that interfaces with the inner shaft. The finger flange 110 is attached at or near its proximal end to the collar 112. Finally, the ablation device 100 also includes a thumb grip 114 at its proximal end, which can be made of an elastomeric material, for example. Such a thumb grip 114 is shown in a relaxed state in FIG. 5.

FIG. 6 illustrates the ablation device 100 with the first and second distal jaws 104, 106 in an open position relative to each other, along with fingers 120 and a thumb 122 of an exemplary human hand, which are holding the jaws 104, 106 in an open position. In order to move the device 110 into this position, a person (e.g., a surgeon) will place at least one finger 120 on the finger flange 110 and thumb 122 on the thumb grip 114. The finger flange 110 can then be slid toward the proximal end of the device 100 by squeezing the fingers 120 toward the thumb 122. When this motion is initiated, the elastomeric finger flange 110 is slid along the outer shaft 102 while the finger flange 110 expands radially outwardly, thereby providing a surface that increases in diameter against which the fingers 120 can press. At the same time, the pressure of thumb 122 on thumb grip 114 can compress the thumb grip 114 so that it also expands radially outwardly, which also provides a surface with a larger diameter for contact with thumb 122. It is further contemplated that compression of the thumb grip 114 can provide a concave surface, which can in turn provide for better contact between the thumb 122 and thumb grip 114. This configuration of an ablation device provides for a relatively clean and unobstructed line of view for the surgeon from the proximal end of the device 100 to its distal end.

A proximal end of an ablation device 140 is illustrated in FIGS. 7 and 8, which operates in a similar manner to the activation end of ablation device 100 described above. FIG. 7 shows the device 140 in its relaxed or inactivated condition, while FIG. 8 shows the device 140 during an activation stage. Ablation device 140 generally includes a shaft 142 on which an elastomeric finger flange 144 is positioned, a spring 146, and a thumb rest 148. In order to move the device 140 toward its activation configuration of FIG. 8, at least one finger is placed at or near the distal end of the flange 144, and then the fingers are brought toward a thumb that is positioned on the thumb rest 148. During this motion, the spring 146 will be compressed as the flange moves along the length of the shaft 142 and the finger flange 144 expands radially outwardly, thereby providing a larger surface for contact with the fingers.

FIG. 9 illustrates an exemplary elastomeric finger flange 150, which can be used with the device 140 of FIGS. 7 and 8, for example, and/or other ablation devices of the invention. Finger flange 150 is generally configured as an elongated tube that includes a series of ribs 152 that are molded into the tube and/or attached to the inside or outside of the tube. These ribs 152 can be provided in a zigzag pattern, as shown, or can have a different configuration. In any case, the ribs 152 can provide structural integrity to the tube in order to prevent the flange from folding over on itself when it is being compressed from its relaxed state to its activated stage. The finger flange 150 can further include one or more concentric rings 154 that can be molded into the tube and/or attached to the inside or outside of the tube. These rings 154 can provide the flange 150 with a surface that is easier to grip with the fingers and move in a consistent manner along a shaft during the activation stage.

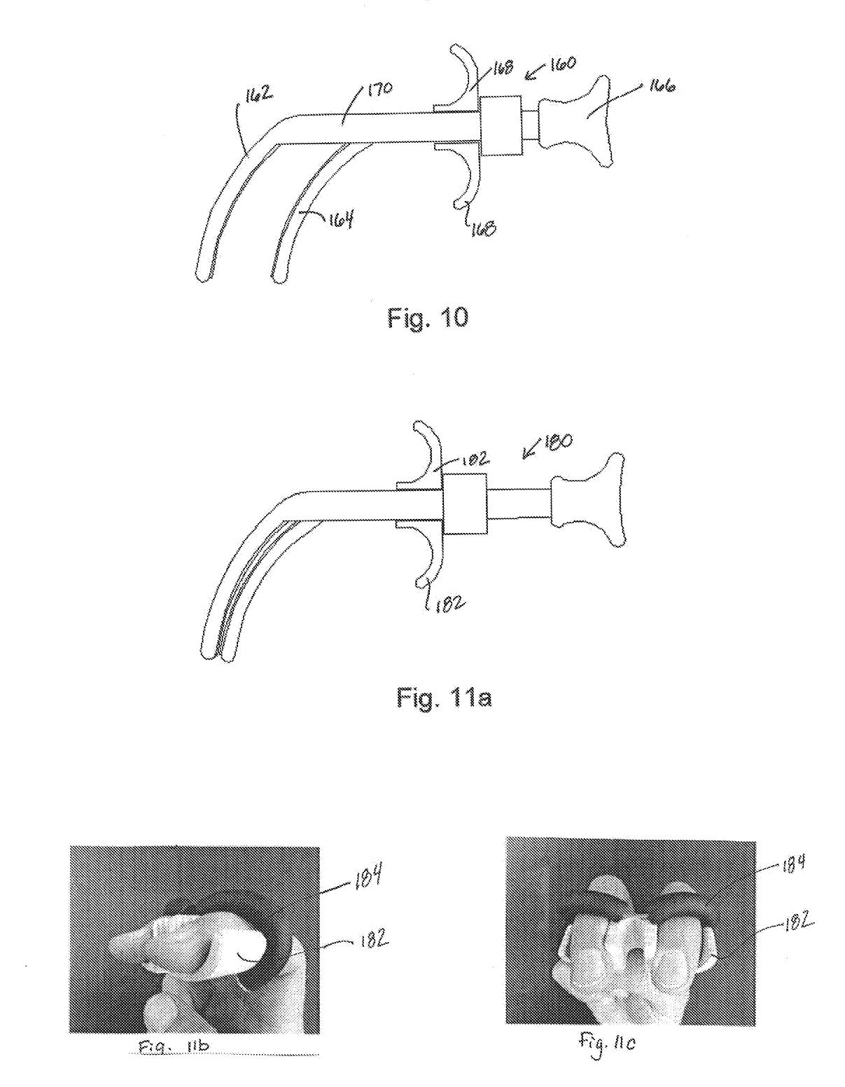

FIG. 10 provides yet another proximal end configuration for activation of distal jaws of an ablation device. In particular, an ablation device 160 is illustrated, which generally includes two distal jaws 162, 164, which are in a closed position when the device 160 is in a relaxed state. Device 160 is further provided with a thumb rest 166 at its proximal end and molded finger grips 168 positioned between the thumb rest 166 and the distal jaws 162, 164. The finger grips 168 are shown as being positioned at an angle of 180 degrees relative to each other, although it is possible that they are arranged at an angle greater or smaller than 180 degrees. Each of the finger grips 168 extends radially relative to a shaft 170 and is provided as a U-shaped extension into which an operator can easily slide a finger. The internal surface of the finger grips 168 can be smooth or textured to provide additional engagement with the surface of a finger. As with other ablation devices described herein, in order to open the distal jaws 162, 164, the finger grips 168 are moved toward the thumb rest 166 by squeezing the fingers toward the thumb until the jaws 162, 164 are moved apart from each other by a sufficient distance. Due to the shape of the finger grips 168, it is also possible for a user to move the fingers toward each other at least slightly in order to move the grips toward the shaft 170 during the jaw opening process.

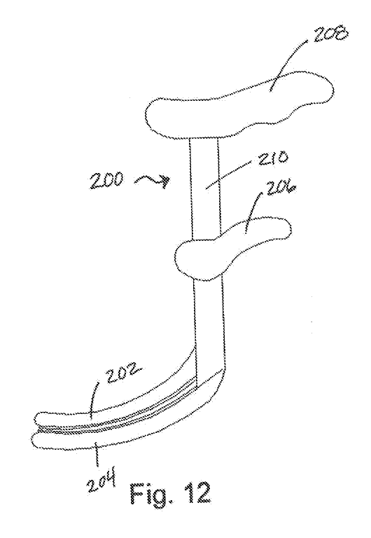

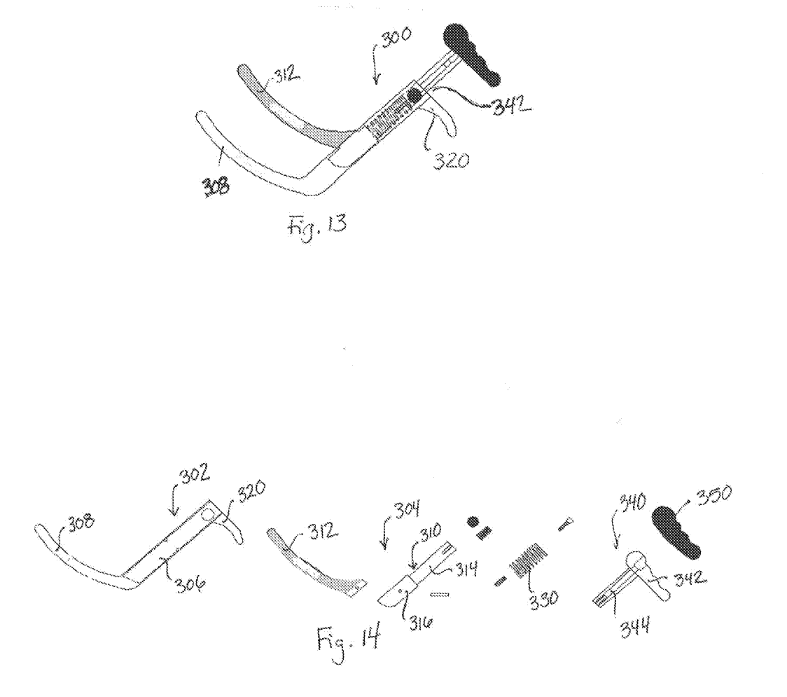

FIGS. 11a-11c illustrate another ablation device 180 that is similar to the device 170 described above, but that further includes at least one finger wrap 184 (not specifically shown in FIG. 11a) that can provide a more secure engagement between the fingers and finger grips 182. Preferably, each finger grip 182 includes a corresponding finger wrap 184. The finger wraps 184 can be made of a bendable material to accommodate fingers of different sizes, and can optionally include a covering material. The finger wraps 184 can have a variety of different lengths; however, each of the finger wraps 184 shown in these figures are long enough to come in contact with or overlap an outer surface of a corresponding finger grip 182, although it is understood that the finger wraps 184 can be shorter such that they do not overlap the finger grips 182. The finger grips 182 and wraps 184 can be anchored to the shaft of an ablation device, or can alternatively be provided as a removable component that can be used with different ablation devices.

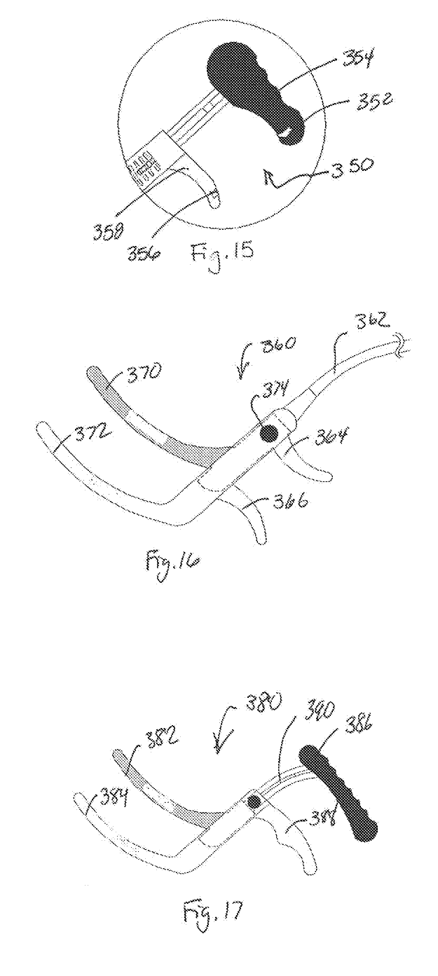

Another embodiment of an ablation device 200 is illustrated in FIG. 12, which generally includes distal jaws 202, 204 that are in a normally closed position when the device has not yet been activated. The device 200 further includes a finger grip 206 that is a molded piece that is positioned around a shaft 210 of the device and that can be used in cooperation with a thumb/palm rest 208 at the proximal end of the device 200. In order to move the distal jaws 202, 204 away from each other and into an open position, the user can place one or more fingers on the bottom side of finger grip 206 and either a palm or thumb on the thumb/palm rest 208, and then pull these two elements toward each other. The thumb/palm rest 208 can have a wide variety of different shapes and sizes to accommodate the desires of the surgeon, the location of the patient in which the device is to be positioned, and a number of other considerations and preferences. This ablation device 200 is provided to position the user's hand away from the center axis of the device to gain additional visibility of the surgical area during placement of the device. In addition, the handle of this device is shifted upward as compared to other embodiments, in order to provide a device that has extra length while being able to minimize the length of shaft 210.

FIGS. 13 and 14 illustrate an exemplary embodiment of an ablation device 300 of the invention, which can be connected to components such as electrical and fluidic connections, as described generally above, although such components are not illustrated in these figures. Ablation device 300 includes a first member 302, which includes an extension member 306 from which a lower jaw 308 extends, and a second member 304, which includes an extension member 310 to which an upper jaw 312 can be attached and extend therefrom. Extension member 310 of second member 304 includes a portion 314 that has a smaller cross-section than a portion 316. In this way, when the device 300 is assembled, a spring 330, which can have an inner diameter that is at least slightly smaller than an outer diameter of portion 316 but slightly larger than an outer diameter of portion 314, can be slid over the portion 314 until it reaches portion 316. The spring 330 will then be unable to slide further along extension member 310 toward the jaw 312, thereby maintaining the spring in this position relative to second member 304. First member 302 further includes a finger flange 320 extending radially outwardly beyond its outer surface.

Second member 304 is positioned relative to first member 302 so that its extension member 310 is generally co-linear and at least partially enclosed within a center lumen of extension member 306. The ablation device 300 further includes a handle 340, which includes a thumb rest 342 from which a post 344 extends. The device may optionally include a cover 350 over at least a portion of the thumb rest 342.

The ablation device 300 is provided with a push-button type of locking mechanism 342 and jaws that are in a normally open position prior to activation. In this embodiment, the first member 302 is a generally tubular structure that is attached to the handle 340, while the upper jaw 312 rides in a proximal and distal direction of the first member 302 inside its tubular structure. Coil spring 330 is also positioned within the tubular structure of member 302 and on the portion 314 of extension member 310. Locking mechanism 342 is spring deployed so that when the jaws 308, 312 come in contact with each other or a plunger reaches the full potential of the spring, a notch or other mechanism can engage with the push button. The push button lock is generally only pushed or activated to release the locking jaws, as opposed to being used to lock the jaws, although it is possible that it is used for both purposes.

The spring 330 is chosen or designed to be capable of controlling force at which the upper and lower jaws close relative to each other. The spring 330 can be capable of being changed or replaced to alter the force of the jaw and actuation force required by pulling on the finger flange 320. That is, spring 330 controls the force that needs to be overcome to move the lower jaw 308 and upper jaw 312 toward each other and into a closed position. Therefore, the spring 330 can be chosen and/or designed to provide a desired actuation force for the user to move the jaws relative to each other

The jaws 308, 312 of ablation device 300 are in a normally open position when the device 300 is not subjected to external forces, with the jaws 308, 312 being generally parallel to each other along at least a portion of their surfaces that face each other. That is, a lower face of jaw 312 would be in contact with at least a portion of an upper face of jaw 308 when the jaws are closed. In order to move these jaws toward each other from their open position, a user would place a thumb on the thumb rest 342 and a finger on a curved surface of finger flange 320, and then squeeze the finger grip 320 toward the thumb rest 342 to slide the first member 302 toward the thumb rest 342, thereby moving the jaws 308, 312 toward each other. When this movement is occurring, the spring 330 will be compressed by a corresponding amount. The spring 330 may be made of spring steel, for example, and may optionally be coated, such as with a PTFE coating.

The ablation device 300 can be a part of a larger system that includes an RF electrode, which may include a porous polymer, hypo-tube, wires, one or more saline lines, and strain-relief members. The various components of the device 300 may include machined components, injection molded components, or components that are made using a variety of different manufacturing techniques. The device 300 may further include additional pins, fasteners, and/or hinges in order to maintain the components in alignment with each other during the ablation process.

FIG. 15 illustrates another embodiment of a proximal end of a handle 350 that can be used with ablation devices of the type described herein, such as the ablation device 300 of FIG. 13. In this embodiment, an alternative or additional locking mechanism is illustrated, which includes a mating latch system that includes a first locking element 352 on a thumb rest 354 and a second locking element 356 on a finger grip 358. The locking elements 352, 356 can be configured so that they engage with each other when pushed together and so that the lock is released or unlatched by pushing the finger grip 358 and thumb rest 354 toward each other again. Alternatively, the lock can be released by pulling the finger grip 358 and thumb rest 354 in opposite directions so that they are released from each other.

Another exemplary ablation device 360 is illustrated in FIG. 16, which includes an electrical connector 362 extending from its proximal end. Because the connector 362 extends from an end of the ablation device 360 that might otherwise include a thumb or palm rest, the second element that can be gripped and squeezed by an operator is instead positioned to extend radially from a shaft of the device 360. In particular, ablation device 360 includes shafts concentrically positioned with one inside the tubular opening of the other, with finger grips 364, 366 extending radially relative to the longitudinal axes of the shafts. Ablation device 360 further includes two extending jaws 370, 372 that are generally parallel to each other along their lengths and that are spaced from each other when the device is in a relaxed or inactivated configuration. In order to move these jaws 370, 372 toward each other, an operator can place a finger in the curved area of each of the finger grips 364, 366 and squeeze them toward each other. This ablation device 360 can optionally include a push-button lock 374, which may be spring deployed, for example, so that when the jaws 370, 372 are pressed into contact with each other or a plunger reaches the full potential of the spring and/or moves to a certain location, a notch or other feature engages with the push button.

An ablation device 380 is illustrated in FIG. 17 that is similar in structure to other ablation devices described herein. Device 380 includes normally open jaws 382, 384 that need to be activated in order be moved toward each other. Device 380 further includes a shaft 390 with a slight curve or bend, which terminates at grip 386 that is provided with at least a slightly larger shape than other described embodiments. Finger grip 388 is also larger than the grips of other described embodiments, and is shown as including two indentations for fingers, although it is understood that finger grip 388 can include more or less than two finger indentations. The curved shape of shaft 390 can provide different visual access to the surgical area as compared to other devices described herein, in that the shape of the shaft removes the operators hand from the center axis of the device. It is further understood that overall device length and corresponding length of various components that make up the device 380 can be relatively longer or shorter than illustrated in the Figure. Device 380 can also include a locking mechanism, such as a push-button lock, which is used to maintain the device in a closed position when external forces are not being applied to the device 380, and/or to keep the device in an open or semi-opened position.

The ablation device 400 illustrated in FIGS. 18 and 19 has many of the same mechanical properties as previously described embodiments, along with similar components and arrangements thereof. Note that FIG. 19 is an exploded view of the ablation device 400, while FIG. 18 illustrates device 400 in its assembled configuration. Device 400 is at least slightly longer and includes a malleable neck section as compared to other embodiments. This malleable neck section provides the capability to bend the neck to allow the surgeon to gain visibility while clamping. The malleable neck section can be relatively small (e.g., one or two inches in length) or can be relatively large relative to the overall neck length, and should be capable of bending to a predetermined radius. For example, the malleable neck section may be capable of bending up to approximately 45 degrees.

The ablation device 400 includes a first member 402 and a second member 404, which components are assembled so that at least a portion of the second member is positioned within an open tubular area of the first member 402. First member 402 includes a jaw 412 and second member 404 includes a jaw 414, both of which extend from distal ends of elongated members of their respective members. Distal jaws 412, 414 are moveable toward and away from each other, and are configured to be in an open position when the device is in its relaxed state. The jaws 412, 414 are configured to be moveable toward each other when the device is activated. Device 400 further includes a thumb rest 406 extending from a proximal end of second member 404, and an incompressible coil 408 extending along a portion of its length. This coil 408 may optionally include a protectable cover layer, such as a PTFE jacket or cover, for example. First member 402 includes a ring-like finger grip 416, which may optionally be replaced with one or more open finger grips of the type described above relative to other ablation device embodiments. First member 402 further includes a malleable section 410 that extends for a portion of the length of the device, and is positioned to generally align with the incompressible coil 408 of second member 404 when the device is assembled, as is illustrated in FIG. 18.

The incompressible coil 408 may be a stainless steel flat-wire coil, which may include a jacket that is heat shrunk onto the coil. The malleable neck section may be made of a corrugated stainless steel, which may also have an outer jacket that is heat shrunk onto the neck section or that is provided as a silicone sleeve, for example. With this embodiment, the flexibilities of the incompressible coil and the malleable neck section can be coordinated to provide overall predetermined flexibility characteristics for the device.

Because the device 400 is in a normally open position when at rest, it will need to be activated or manipulated in order to put the jaws 412, 414 in contact with each other. In order to accomplish this, the thumb rest 406 is moved relative to the finger grip 416, during which the coil 408 will move up and down within the malleable neck section 410. The incompressible coil 408 provides on-axis rigidity for actuation of the device, while simultaneously allowing for the neck to have at least slight malleability. The device 400 may include a locking mechanism, if desired, to keep the components in a desired position relative to each other.

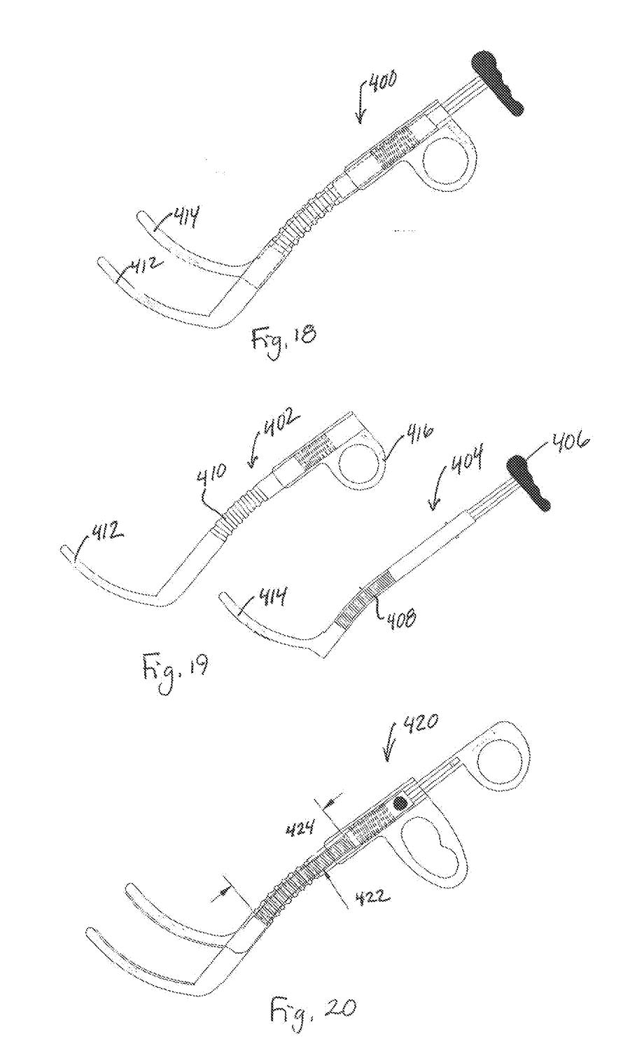

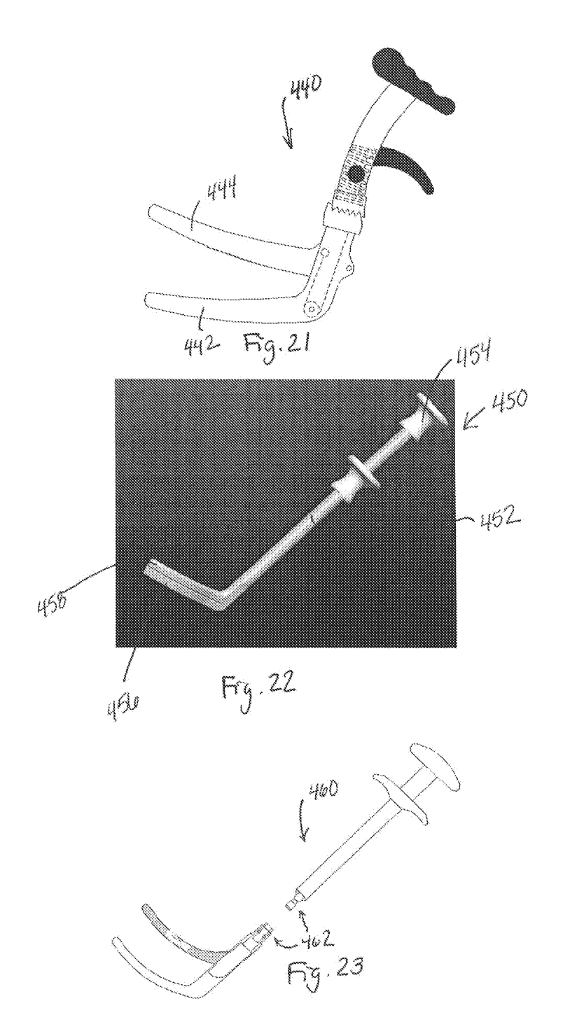

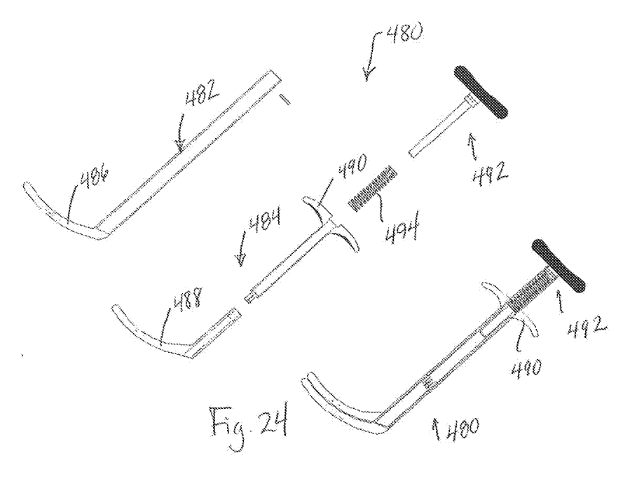

FIG. 20 illustrates another ablation device 420 that is configured similarly to the device 400 of FIGS. 18 and 19; however, ablation device 420 includes a longer incompressible coil section 422 (the length of which is designated by reference number 424), which may be longer than a corresponding malleable section of the device. This neck will remain at least semi-malleable at all times, no matter where the device is in its actuation cycle. In addition, device 420 includes ring-like finger grips for both of its first and second members, although different shaped and sized finger grips are contemplated and are within the scope of the invention.

Another ablation device embodiment 440 is illustrated in FIG. 21, which includes many of the same components described above relative to other ablation devices of the invention. The ablation device 440 includes non-parallel jaws that are in a normally open configuration when not subjected to external forces. This device 440 can include a somewhat shorter neck than ablation device 400, for example, and can include a push-button lock. As shown, a lower jaw 442 is fixed to the main heel structure and an upper jaw 444 is controlled by way of a cable and roller system. The optional lock mechanism can be spring deployed such that it is engaged once both jaws 442, 444 are closed relative to each other, or so that it is engaged when the lower finger grip moves its farthest distance where a notch becomes engaged with the push button. A main coil spring will also be used to control the force at which the upper and lower jaws close together. This spring can be altered in a number of different ways in order to alter the force of the jaws and the actuation force of the finger grips.

FIG. 22 illustrates another ablation device 450, which includes a number of the features and configurations described above relative to normally closed ablation devices of the invention. This device 450 includes a neck section 452 that is relatively long as compared to those discussed herein for other embodiments. This longer neck section 452 provides a configuration with which the surgeon gains visibility while clamping and to reach all lesion locations. A top plunger 454 is actuated to open the jaws 456, 458. This embodiment of an ablation device provides for a “direct drive” type of actuation, which can be relatively easy to maneuver and activate by a surgeon.

FIG. 23 illustrates a feature for ablation devices that can be used with many of the ablation device configurations described herein. In particular, an ablation device 460 is illustrated, which includes a quick-connect/quick-disconnect coupling 462. Such a coupling can include a snap-fit connection, for example, and can provide for significant flexibility for interchanging components of various ablation devices. For example, if a surgeon prefers a particular activation mechanism at a proximal end of the device or prefers a certain malleability for a device, while also requiring a particular jaw configuration, the surgeon can select components having the desired features and easily fit them together to make a customized device. In such a case, it is possible that the ablation device is provided to the surgeon as a kit that includes a variety of different components.

FIG. 24 illustrates an ablation device 480 with assembled and exploded views, wherein this device 480 can be connected to components such as electrical and fluidic connections, as described generally above, although such components are not illustrated in these figures. Ablation device 480 includes a first member 482, and a second member 484 that is linearly moveable relative to the first member 482. In particular, first member 482 includes an extension member from which a jaw 486 extends, while second member 484 includes an extension member from which a jaw 488 extends. The first member 484 includes a quick-connect/quick-disconnect coupling, such as is described above relative to FIG. 23. Device 480 is further provided with a spring 494 that is positioned between the first and second members 482, 484 when the device is assembled. Second member 484 further includes a finger grip 490 that extends radially at its distal end. The device further includes a handle 492 at its proximal end, which can include a member against which a thumb or palm can be pressed to compress a spring 494 to move the jaws 486, 488 relative to each other.

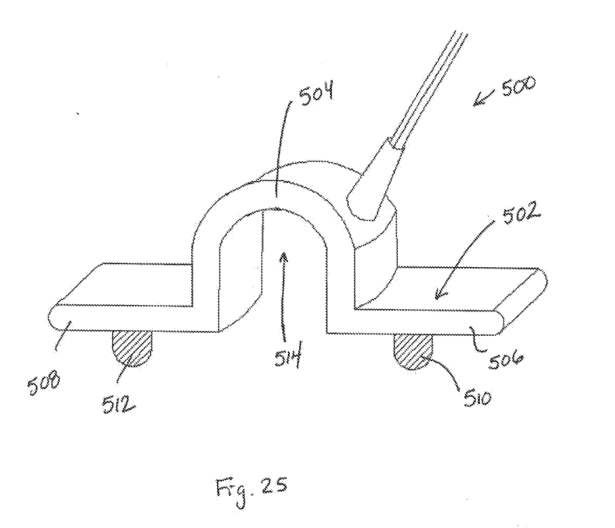

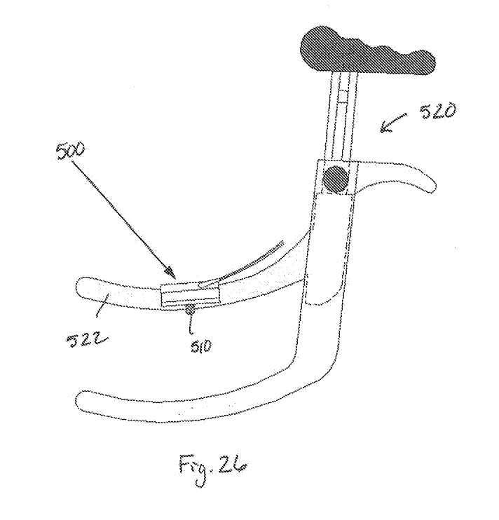

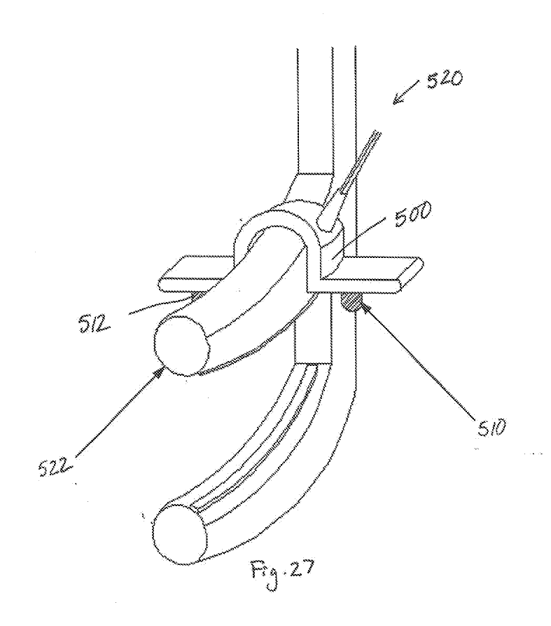

FIG. 25 is a perspective view of a clip-on sensing apparatus 500 of the invention, which is configured for attaching to a number of different devices, such as a standard bipolar ablation jaw, an ablation device of the type described above, and/or other devices or apparatuses that can utilize such a sensing apparatus. The sensing apparatuses of the invention allow an operator to pace or sense for confirmation of conduction block in a patient. In particular, during creation of a bipolar ablation line or after such a line is created, a surgeon can pace or sense with the sensing apparatus on both sides of a created ablation line to confirm conduction block while leaving the ablation jaws and the device in position within the patient. If feedback from the sensing apparatus indicates that conduction block has not been achieved by the ablation procedure, the surgeon can then optionally perform one or more additional ablation procedures until a complete line of block is created.

A sensing apparatus of the invention can have monopolar or bipolar electrodes for pacing and/or sensing. The electrodes can optionally be spring loaded or otherwise repositionable so that they can collapse or otherwise be reoriented while ablation is taking place in order to not interfere with the ablation process. The apparatus can further include side tabs for holding the electrodes, which can also serve as spring tabs. The sensing/pacing electrodes can be made of a variety of materials, such as electrodes that are 80% platinum and 20% iridium in order to provide noise-free signals, for example. The use of one or more sensing apparatuses of the invention can eliminate the need to use a secondary instrument to check for conduction block after ablation, which can be particularly beneficial when the ablation is being performed in an area of the body that is difficult to reach and/or when it is useful to monitor live conduction signals.

In accordance with the invention, a sensing apparatus can be designed to provide a low effort clip-on design. Attachment of the apparatus to an associated device can be accomplished in a number of ways, such as via a clamping mechanism or with a snap-fit connection, for example. The apparatus can include any shape or size that best fits the device positioning and does not impede with the placement of an instrument to which it is attached, such as a bipolar ablation instrument. The pacing/sensing electrodes of the device can be provided in any configuration, including monopolar or bipolar pairs, for example, or can include multiple electrodes on one or both sides of the apparatus in order to be able to observe the entire cardiac cycle potentials and refractory periods. In use, this apparatus provides a surgeon with the ability to check entrance and exit block of isolated areas of the heart. It is also contemplated that multiple clip-con pacing/sensing apparatuses of the invention can be used on a single instrument, such as on an ablation device of the type described herein and/or other devices. With ablation devices of the type having upper and lower jaws, as described herein, one sensing apparatus can be attached to the upper jaw and another one can be attached to the lower jaw, for example. Any sensing/pacing apparatuses can be either permanently or removably attached to the desired device, and can be repositionable for optimum placement of the apparatus relative to the device to which it is attached.

As is illustrated relative to the embodiment of FIG. 25, sensing apparatus 500 includes frame 502 and two pacing/sensing electrodes 510, 512. In this embodiment, frame 502 includes a generally U-shaped member 504 with a first member 506 extending from one distal end of the U-shaped member 504 and a second member 508 extending from the other distal end of the U-shaped member 504 and in an opposite direction from the first member 504. Each of the first and second members 506, 508 includes one of the electrodes 510, 512 extending outwardly from one of its surfaces (e.g., extending from a lower surface relative to the illustrated figure). It is contemplated that the U-shaped member 504 can instead have a shape other than a “U” in order to accommodate the shape of a device to which it will be attached. However, the illustrated embodiment includes an inner area 514 of member 504 that is defined by a surface that is curved to correspond with a curved outer surface of a member to which it will be attached.

The sensing apparatus 500 is illustrated in an exemplary configuration relative to an ablation device 520 in FIGS. 26 and 27, wherein the apparatus 500 is attached to an upper jaw 522 of device 520. One or both of the electrodes 510, 512 are visible in these drawings, and as illustrated, are spaced at a distance from the upper jaw 522, wherein these electrodes are positioned for sensing at desired locations relative to an ablation line that will be created by the ablation device.

In addition to materials discussed above, the ablation devices of the invention can be made from a wide variety of materials, such as porous media, hypotube, coated metals, uncoated metals, combinations of materials, and/or other materials approved for use in surgical procedures. Embodiments can include internal thermocouples for monitoring the temperature before, during, and after ablation. It is further noted that in embodiments that include an axial spring, the spring force can be adjustable for transmurality on myocardial tissue.

In accordance with embodiments of the invention, the jaw subassemblies may be used as a monopolar device, as well as a device having one inactive jaw. The devices of the invention may be irrigated or non-irrigated, wherein when the devices are irrigated, they can be associated with an irrigation source that can deliver irritation fluid to the ablation site. The irrigation source can be manually controllable, such as with a switch that can be turned on and off by a surgeon or other operator, or can be designed to automatically start and stop in response to certain input received during the ablation process, wherein various irrigation processes, fluids, and controls are discussed in U.S. Pat. No. 6,887,238, the entire disclosure of which is incorporated herein by reference.

It is further contemplated that the devices of the invention can deliver ablation via a number of different energy sources, such as such as can be provided by RF sources, microwave, ultrasound, pulsed RF, electroporation, and the like, wherein a number of different energy sources that can be used are disclosed in U.S. Pat. No. 6,887,238, the entire disclosure of which is incorporated herein by reference. It is further contemplated that although the disclosure herein is directed primarily to use of the devices in open chest types of surgical procedures, it is understood that the devices can also be used in minimally invasive surgical procedures, along with those that are controlled robotically.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

What is claimed is:

1. An ablation device for use in a surgical procedure, comprising:
a first member comprising a first elongated shaft and a first jaw extending from a distal end of the first elongated shaft;
a second member comprising a second elongated shaft and a second jaw extending from a distal end of the second elongated shaft, wherein the second elongated shaft is at least partially positioned within an inner tubular opening of the first elongated shaft, and wherein the second jaw has a bottom surface that is generally parallel to at least a portion of a top surface of the first jaw;
a handle extending from a proximal end of the first elongated shaft; and
a finger grip extending radially outward relative to an outer surface of the first elongated shaft.

2. The ablation device of claim 1, further comprising a spring positioned between an outer surface of the second elongated shaft and an inner surface of the first elongated shaft.

3. The ablation device of claim 2, wherein the first and second jaws are in a closed configuration when the ablation device is not subjected to external compression forces.

4. The ablation device of claim 2, wherein the first and second jaws are moveable by compression of the spring to an open position that comprises a space between the bottom surface of the second jaw and the top surface of the first jaw.

5. The ablation device of claim 1, wherein the handle comprises a shaft and a thumb rest extending from a proximal end of the shaft.

6. An ablation device for use in a surgical procedure, comprising:
a first member comprising a first elongated shaft and a first jaw extending from a distal end of the first elongated shaft;
a second member comprising a second elongated shaft and a second jaw extending from a distal end of the second elongated shaft, wherein the second elongated shaft is at least partially positioned within an inner tubular opening of the first elongated shaft, and wherein the second jaw has a bottom surface that is generally parallel to at least a portion of a top surface of the first jaw;
a thumb rest extending from a proximal end of the first elongated shaft; and
a tubular finger flange at least partially surrounding an outer surface of the first elongated shaft, wherein a distal end of the finger flange is slideable toward a proximal end of the first elongated shaft while the finger flange simultaneously expands radially outwardly.

7. The ablation device of claim 6, wherein the finger flange comprises an elastomeric material.

8. The ablation device of claim 6, wherein the finger flange comprises a plurality of ribs located around its perimeter.

9. The ablation device of claim 6, wherein the finger flange comprises a plurality of support rings spaced from each other along at least a portion of a length of the finger flange.

10. An ablation device for use in a surgical procedure, comprising:
a first member comprising a first elongated shaft and a first jaw extending from a distal end of the first elongated shaft;
a second member comprising a second elongated shaft and a second jaw extending from a distal end of the second elongated shaft, wherein the second elongated shaft is at least partially positioned within an inner tubular opening of the first elongated shaft, and wherein the second jaw has a bottom surface that is generally parallel to at least a portion of a top surface of the first jaw;
a thumb rest extending from a proximal end of the first elongated shaft; and
a finger grip member extending radially from and adjacent to an outer surface of the first elongated shaft, wherein the finger grip comprises at least one finger engagement portion spaced longitudinally from the thumb rest.

11. The ablation device of claim 10, wherein the finger grip member comprises a first finger engagement portion positioned approximately 180 degrees from a second finger engagement portion.

12. The ablation device of claim 10, wherein the finger grip member comprises a flexible finger wrap associated with each finger engagement portion for wrapping around an upper surface of a finger of a user.

13. The ablation device of claim 10, wherein the finger grip member comprises at least one U-shaped finger engagement member.
